(19) United States
(12) Patent Application Publication
(10) Pub. No.: US 2023/0330333 A1
(43) Pub. Date: Oct. 19, 2023

(54) DRIVING STRUCTURE OF A DRUG INFUSION DEVICE

(71) Applicant: MEDTRUM TECHNOLOGIES INC., Shanghai (CN)

(72) Inventor: Cuijun YANG, Shanghai (CN)

(73) Assignee: MEDTRUM TECHNOLOGIES INC., Shanghai (CN)

(21) Appl. No.: 18/034,070

(22) PCT Filed: Nov. 27, 2020
(86) PCT No.: PCT/CN2020/132037

§ 371 Date: Apr. 27, 2023

(51) Int. Cl. A61M 5/172; A61M 5/142

(52) U.S. Cl. CPC A61M 5/172; A61M 5/142; A61M 2205/82; A61M 2005/14208

(57) ABSTRACT

A driving structure of a drug infusion device, includes at least one driving unit and at least one driving wheel; a linear actuator, electrically connected with the driving unit, pulling the driving unit to move in the driving direction after being powered; a power supply used to supply the power to the linear actuator; a program unit and a first switch unit. The power supply, the program unit, the first switch unit and the linear actuator are electrically connected, when the linear actuator is powered, the driving unit implements driving, and the driving unit triggers a first signal, indicating the end point of the driving direction, which reduces the power consumption of the infusion device.

### TECHNICAL FIELD

The present invention mainly relates to the field of medical instruments, in particular to a driving structure of a drug infusion device.

### BACKGROUND

The pancreas in a normal person can automatically monitor the amount of glucose in the blood and automatically secrete the required dosage of insulin/glucagon. However, for diabetic patients, the function of the pancreas is abnormal, and the pancreas cannot normally secrete required dosage of insulin. Therefore, diabetes is a metabolic disease caused by abnormal pancreatic function and also a lifelong disease. At present, medical technology cannot cure diabetes, but can only control the onset and development of diabetes and its complications by stabilizing blood glucose.

Patients with diabetes need to check their blood glucose before injecting insulin into the body. At present, most of the detection methods can continuously detect blood glucose, and send the blood glucose data to the remote device in real time for the user to view. This detection method is called Continuous Glucose Monitoring (CGM), which requires the detection device to be attached to the surface of the patients' skin, and the sensor carried by the device is inserted into the subcutaneous tissue fluid for testing. According to the blood glucose (BG) level, the infusion device, as a closed-loop or semi-closed-loop artificial pancreas, injects the currently required insulin dose.

However, the current driving structure of the drug infusion device has a relatively large power consumption, which raises the requirements on the power supply, and has poor reliability.

Therefore, the prior art urgently needs a drug infusion device with low power consumption and high reliability.

### BRIEF SUMMARY OF THE INVENTION

The embodiment of the present invention discloses a driving structure of a drug infusion device. The program unit and the first switch unit control the power to the linear actuator, but only the first switch unit cuts off the power to the linear actuator. The response speed of the first switch unit is faster than that of the program unit, reducing the power consumption of the infusion device.

The invention discloses a driving structure of a drug infusion device, which comprises: at least one driving unit and at least one driving wheel, the driving unit, moving in the driving direction, can drive the driving wheel to rotate; a linear actuator, electrically connected with the driving unit, pulling the driving unit to move in the driving direction after being powered; a power supply used to supply the power to the linear actuator; a program unit and a first switch unit, the power supply, the program unit, the first switch unit and the linear actuator are electrically connected to form a power supply circuit, when the linear actuator is powered, the driving unit implements driving, and the driving unit can trigger a first signal, indicating the end point of the driving direction, which controls the first switch unit to turn off to cut off the power to the linear actuator.

According to one aspect of the present invention, the driving unit includes at least one driving portion, the driving wheel is provided with wheel teeth which can be pushed by the driving portion to drive the driving wheel.

According to one aspect of the present invention, the movement mode of the driving unit includes linear reciprocating movement or rotary reciprocating movement.

According to one aspect of the present invention, it further includes an elastic member, which applies a resetting and resilience force to the driving unit, and the elastic member cooperates with the linear actuator to make the driving unit reciprocate.

According to one aspect of the present invention, it further includes an electrical contact point, which is used to determine the end point of the movement of the driving unit in the driving direction, and the driving unit contacts the electrical contact point to trigger a first signal which is an electrical signal.

According to one aspect of the present invention, the electrical contact point is electrically connected with the first switch unit to form a connection circuit, and the electrical signal controls the first switch unit to turn off.

According to one aspect of the present invention, it further includes a controller, which is electrically connected to the first switch unit and the electrical contact point, respectively, and the electrical signal controls the first switch unit to turn off through the controller.

According to one aspect of the present invention, before and after the driving unit contacts the electrical contact point, the voltage of the electrical contact point is different, and the electrical signal is a voltage changing signal.

According to one aspect of the present invention, the program unit is electrically connected with the electrical contact point to receive the electrical signal.

According to one aspect of the present invention, the program unit includes a timer which controls to disconnect the power supply circuit, and when the linear actuator starts to be powered, the timer starts timing, after the timer goes off a period of time T, if the time from the linear actuator being powered to triggering the first signal is t, then T≥t.

According to one aspect of the present invention, 0 ms≤T−t≤30 ms.

According to one aspect of the present invention, it further includes a second switch unit, which is arranged on the power supply circuit, and controlled to disconnect the power supply circuit by the program unit.

According to one aspect of the present invention, it further includes an electrical connection point located on the power supply circuit between the linear actuator and the first switch unit, and the program unit is electrically connected to the electrical connection point to obtain a second signal, a voltage changing signal, of the electrical connection point.

According to one aspect of the present invention, it further includes a pressure sensor, the pressure sensor is used to determine the end point of the movement of the driving unit in the driving direction, and the first signal is a pressure changing signal.

According to one aspect of the present invention, the first switch unit or the second switch unit includes a MOS field effect transistor, an analog switch or a relay.

According to one aspect of the present invention, the linear actuator is a shape memory alloy.

Compared with the prior art, the technical solution of the present invention has the following advantages:

In the drive structure of the drug infusion device disclosed in the present invention, the power supply, the program unit, the first switch unit and the linear actuator are electrically connected to form a power supply circuit, when the linear actuator is powered, the driving unit implements driving, and the driving unit can trigger a first signal, indicating the end of the driving direction, which controls the first switch unit to turn off to cut off the power to the linear actuator. The response speed of the first switch unit is faster than that of the program unit. Compared with the power to the linear actuator controlled by the program unit simultaneously, the first switch unit can cut off the power to the linear actuator much more quickly, shortening the powered time to the linear actuator, thereby reducing the power consumption of the infusion device. In addition, shortening the powered time to the linear actuator also reduces the probability of fatigue fracture of the linear actuator, improving the safety of the driving and the infusion reliability of the infusion device.

Furthermore, the elastic member applies a resetting and resilience force to the driving unit, and the elastic member cooperates with the linear actuator to make the driving unit reciprocate. The elastic member can reset the driving unit automatically without consuming electric energy, which further reduces the power consumption of the infusion device.

Furthermore, it further includes an electrical contact point, which is used to determine the end point of the movement of the driving unit in the driving direction, and the driving unit contacts the electrical contact point to trigger a first signal which is an electrical signal. The electrical signal received by the program unit can not only be used to control the disconnection of the power supply circuit to the linear actuator, but also to record the number of power-on and power-off times of the linear actuator, optimizing the control process of the program unit.

### BRIEF DESCRIPTION OF THE DRAWINGS

FIG. 1 is a schematic diagram of the connection relationship between the unit modules of the driving structure of a drug infusion device according to an embodiment of the present invention;

FIG. 2 is a partial schematic diagram of a driving structure according to an embodiment of the present invention;

FIG. 3 is a partial schematic diagram of a driving structure according to another embodiment of the present invention;

FIG. 4a-FIG. 4c are schematic diagrams of the control circuit of a driving structure according to an embodiment of the present invention;

FIG. 5a-FIG. 5c are schematic diagrams of the control circuit of a driving structure according to another embodiment of the present invention;

FIG. 6a-FIG. 6b are schematic diagrams of the control circuit of a driving structure according to another embodiment of the present invention.

### DETAILED DESCRIPTION

As mentioned above, the power consumption in the prior art infusion device is relatively large.

After research, it is found that the reason for the above problems is that the linear actuator is fully controlled by the program unit to be powered on or off. Because the program unit needs a longer response time, the linear actuator takes longer to be powered on, thereby consuming more power.

In order to solve this problem, the present invention provides a driving structure of a drug infusion device. The program unit and the first switch unit control the power to the linear actuator while only the first switch unit cuts off the power to the linear actuator. The response speed of the first switch unit is faster than that of the program unit, reducing the power consumption of the infusion device.

Various exemplary embodiments of the present invention will now be described in detail with reference to the drawings. The relative arrangement of the components and the steps, numerical expressions and numerical values set forth in the embodiments are not to be construed as limiting the scope of the invention.

In addition, it should be understood that, for ease of description, the dimensions of the various components shown in the figures are not necessarily drawn in the actual scale relationship, for example, the thickness, width, length or distance of certain units may be exaggerated relative to other structures.

The following description of the exemplary embodiments is merely illustrative, and is not intended to be in any way limiting the invention and its application or use. The techniques, methods and devices that are known to those of ordinary skill in the art may not be discussed in detail, but such techniques, methods and devices should be considered as part of the specification.

It should be noted that similar reference numerals and letters indicate similar items in the following figures. Therefore, once an item is defined or illustrated in a drawing, it will not be discussed further in following description of the drawings.

FIG. 1 is a schematic diagram of the connection relationship between the unit modules of the driving structure of the drug infusion device according to the embodiment of the present invention.

The drug infusion device includes an infusion needle, a drug storage unit, a piston arranged in the drug storage unit, a screw connected with the piston, a driving structure, and the like. The advancement of the screw can directly push the piston forward to achieve drug infusion.

The driving structure of the drug infusion device includes a power supply, a program unit, a first switch unit, a linear actuator, at least one driving unit and at least one driving wheel.

The power supply is used to supply the power to the linear actuator. The linear actuator and the driving unit are electrically connected to each other. The power supply, the program unit, the first switch unit and the linear actuator are electrically connected to form a power supply circuit for supplying the power to the linear actuator.

It should be noted that the embodiment of the present invention does not limit the order of electrical connection of the first switch unit and the program unit. Preferably, in the embodiment of the present invention, the program unit, the first switch unit and the linear actuator are electrically connected in sequence. In another embodiment of the present invention, the first switch unit, the program unit and the linear actuator are electrically connected in sequence.

The program unit is used to control certain functional units in the infusion device to perform corresponding functions, such as including but not limited to directly controlling the power to the linear actuator or disconnecting the above-mentioned power supply circuit, detecting the amount of the remaining drug, warning, and priming the infusion needle.

In an embodiment of the present invention, the driving device further includes a second switch unit, which is arranged on the power supply circuit. The program unit controls the connection and disconnection of the power supply circuit by controlling the turn-on and turn-off of the second switch unit. The working principle of the second switch unit is similar to that of the first switch unit, which will be described in detail below.

In the embodiment of the present invention, the first switch unit is used to cut off the power to the linear actuator, making the driving unit stop driving the driving wheel. The first switch unit or the second switch unit includes MOS field effect transistor, an analog switch or a relay. Preferably, in the embodiment of the present invention, the first switch unit is a MOS field effect transistor, which controls the conduction and disconnection of the channel according to the voltage change applied to the gate, thereby realizing the connection and disconnection of the power supply circuit. The second switch unit is an analog switch.

What needs to be explained here is that “cutting off the power to the linear actuator” and “disconnecting the power supply circuit to the linear actuator” have completely different meanings. “Cutting off the power to the linear actuator” refers to directly removing the power supplied to the linear actuator, thereby stopping applying the driving force to the driving unit. For example, the turning off of the first switch unit directly stops the linear actuator from being powered. However, “disconnecting the power supply circuit to the linear actuator” only refers to disconnecting the power supply circuit, that is, not necessarily directly remove the power to the linear actuator. For example, the program unit as described below controls the disconnection of the power supply circuit.

The linear actuator is used to apply driving force to the driving unit for movement. When powered on, the physical form of the material of the linear actuator changes, which makes shrinkage deformation of the linear actuator occur, thereby exerting the driving force to move the driving unit. The higher the current is, the larger the shrinkage deformation of the linear actuator occurs, and the greater the driving force outputs. Obviously, when the current is constant, the driving force output by the linear actuator is also constant, because of which the linear actuator can output stable and controllable infusion driving force.

Preferably, the linear actuator is an electrically driven linear actuator or an electrically heated linear actuator. By alternately being powered on and off, the linear actuator outputs power in pulses. Preferably, in the embodiment of the present invention, the linear actuator is a shape memory alloy.

The driving unit can drive the driving wheel to rotate, thereby realizing drug infusion. In the embodiment of the present invention, the driving unit includes at least one driving portion (like 151 in FIG. 2), and wheel teeth are provided on the driving wheel. Therefore, the driving portion can push the wheel teeth to drive the driving wheel to rotate, thereby driving the screw to advance. Preferably, in the embodiment of the present invention, a driving portion is provided on the driving unit, and the driving wheel is a ratchet wheel with ratchet teeth. The ratchet teeth can be pushed more easily, improving driving efficiency.

The movement mode of the driving unit includes linear reciprocating movement or rotary reciprocating movement. Preferably, in the embodiment of the present invention, the driving unit rotates reciprocating around a fixed shaft. The driving structure is also provided with an elastic member (like 170 in FIG. 2) for applying a resetting and resilience force to the driving unit. When the driving unit moves in the driving direction, the elastic member exerts an ever-increasing resetting and resilience force to the driving unit. The elastic member can reset the driving unit automatically without consuming electric energy which reduces the power consumption of the infusion device. Preferably, the elastic member is a spring.

Under the cooperation of the elastic member and the linear actuator, the driving unit performs a rotary reciprocating movement. And the driving unit can push the wheel teeth when it moves in the driving direction, and it stops pushing the wheel teeth while moving in the resetting direction, which will be described in detail below in conjunction with FIG. 2.

In other embodiments of the present invention, the driving unit may further include two or more driving portions. Different driving portions can be driven in cooperation with different driving wheels. At this time, the linear actuator can pull the driving unit to push the wheel teeth in the two directions of reciprocating rotation respectively, making the driving wheel rotate. Therefore, there is no need to provide an elastic member.

In other embodiments of the present invention, the driving unit may also be a gear cooperating with the driving wheel, which is not specifically limited herein.

Generally, after moving for a certain distance, the driving unit needs to stop moving. Therefore, in order to determine the end point of the movement of the driving unit in the driving direction, the driving unit needs to trigger a first signal indicating that it reaches the end point of the driving direction. The first signal is used to control the first switch unit to turn off to cut off the power to the linear actuator, which will be described in detail below.

FIG. 2 is a partial schematic diagram of a driving structure according to an embodiment of the present invention.

In the embodiment of the present invention, when the linear actuator 180 pulls the driving unit 150 by force F_{P}, the driving unit 150 rotates counter-clockwise around the rotating shaft 160 to push the wheel teeth 141 forward to drive the driving wheel 140 rotate, thereby driving the screw 130 to advance in the D_{A} direction. At this time, the elastic member 170 generates an ever-increasing resetting and resilience force F_{R}. The driving structure is also provided with an electric contact point 171 for determining the end point of the movement of the driving unit 150 in the driving direction. Therefore, the contact between the driving unit 150 and the electric contact point 171 will trigger an electric signal.

The electrical contact point 171 is directly electrically connected to the first switch unit to form a connection circuit. When the driving unit 150 is in contact the electrical contact point 171, the voltage of the electrical contact point 171 changes. The voltage changing signal directly turns off the first switch unit, thereby cutting off the power to the linear actuator 180, and directly making the linear actuator 180 stop providing power to the driving unit 150. Thus, the driving unit 150 resets and rotates clockwise around the shaft 160 under the elastic force F_{R}.

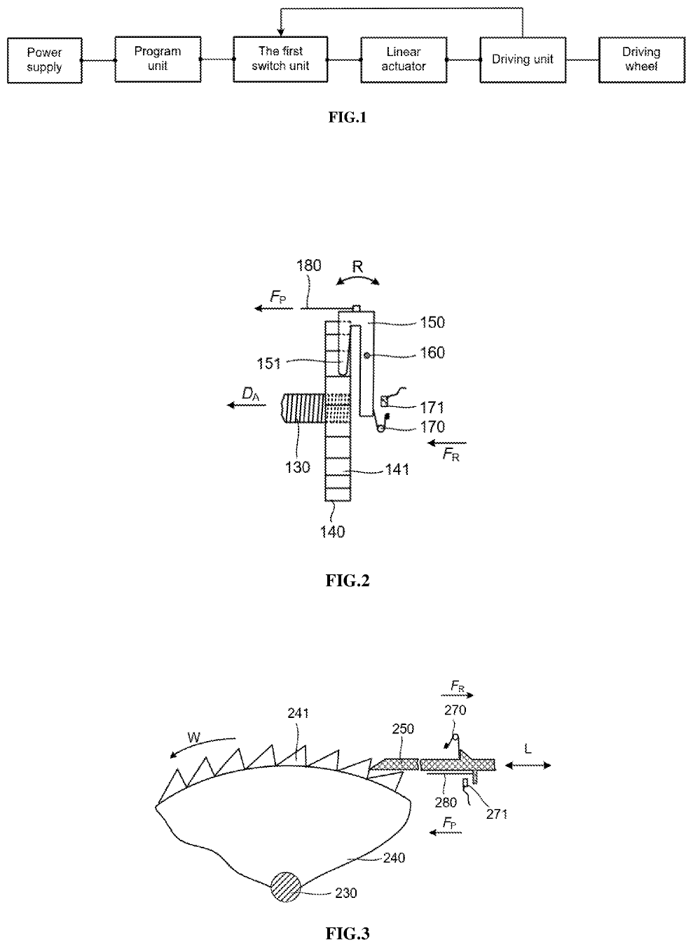

FIG. 3 is a partial schematic diagram of a driving structure according to another embodiment of the present invention.

The linear actuator 280 and the elastic member 270 act on the driving unit 250 with the force F_{P} and F_{R}, respectively, making the driving unit 250 linearly reciprocate in the L direction. Therefore, the driving unit 250 can push the wheel teeth 241 in the driving direction, making the driving wheel 240 move in W direction to realize drug infusion.

Similarly, the electrical contact point 271 is provided in the driving structure. After the driving unit 250 moving in the linear driving direction and contacting the electrical contact point 271, the first switch unit is turned off to cut off the power to the linear actuator 280, making the driving unit 250 stop moving, and then resetting under the force of the elastic member 270. The driving principle is similar to the foregoing said, which will not repeat herein.

In yet another embodiment of the present invention, the pressure sensor is used to determine the end point of the movement of the driving unit in the driving direction. Therefore, the first signal is a pressure changing signal.

Similarly, in other embodiments of the present invention, the first electrical signal is not limited to an electrical signal or a pressure changing signal, and can also be other signals well known to those skilled in the art, as long as it can satisfy the condition for determining the end point of the movement of the driving unit in the drive direction.

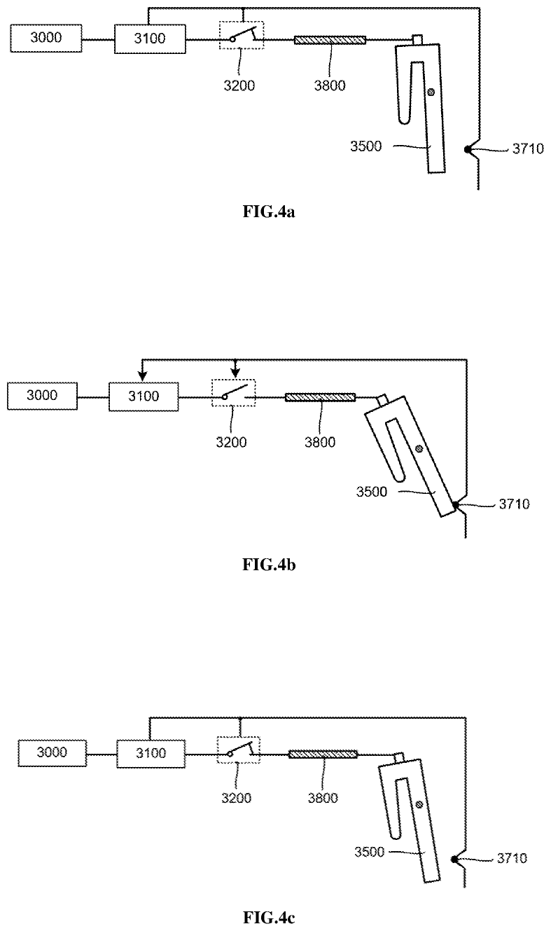

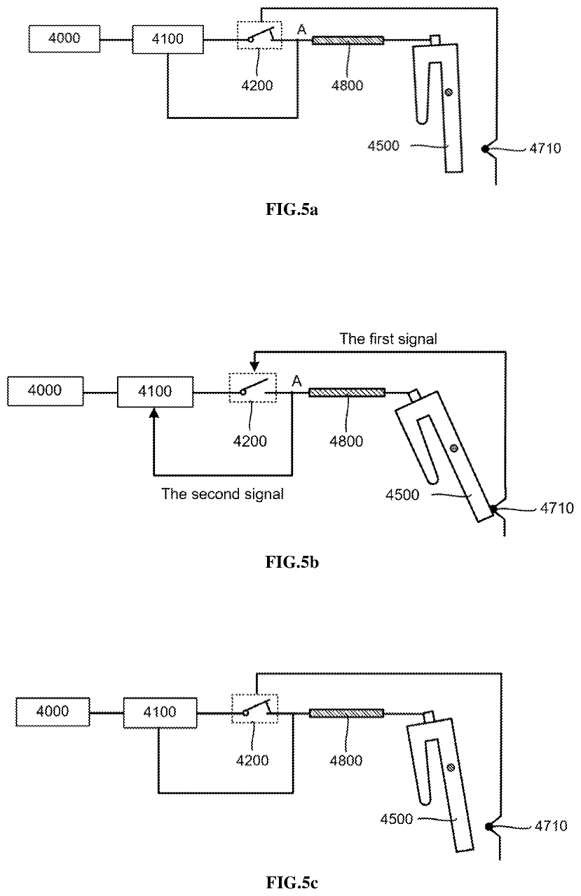

Hereinafter, the circuit control principle of the driving structure will be described by taking the rotating reciprocating movement of the driving unit as an example. The elastic members are not shown in FIG. 4a-FIG. 6b.

FIG. 4a-FIG. 4c are schematic diagrams of the control circuit of the driving structure of the embodiment of the present invention.

As shown in FIG. 4a, the electrical contact point 3710 is electrically connected to the first switch unit 3200 to form a connection circuit. When the driving unit 3500 is not in contact with the electrical contact point 3710, the driving unit 3500 is at a low voltage state while the electrical contact point 3710 is at a high voltage state. When the linear actuator 3800 needs to work, the program unit 3100 and the first switch unit 3200 control the power from the power supply 3000 to the linear actuator 3800, thus, the driving unit 3500 starts driving, that is, the driving unit 3500 moves in the driving direction (eg., turning counter-clockwise).

As shown in FIG. 4b, when the driving unit 3500 is in contact with the electrical contact point 3710, the voltage of the electrical contact point 3710 changes, such as from a high voltage state to a low voltage state. The voltage changing signal can immediately control the first switch unit 3200 to turn off. In the embodiment of the present invention, the program unit 3100 is also electrically connected with the electrical contact point 3710 to receive the voltage changing signal, and can control to disconnect the power supply circuit.

Obviously, the program unit 3100 and the first switch unit 3200 can simultaneously receive the voltage changing signal, and both can disconnect the power supply circuit. However, generally, the response speed of the first switch unit 3200 is much faster than that of the program unit 3100. Therefore, the first switch unit 3200, not the program unit 3100, cuts off the power to the linear actuator 3800.

As shown in FIG. 4c, when the driving unit 3500 leaves the electrical contact point 3710 under the force of the elastic member, the electrical contact point 3710 returns to the high voltage state, and the first switch unit 3200 will turn on again. Since the program unit 3100 controls to disconnect the power supply circuit, the linear actuator 3800 still cannot be powered again, making the driving unit 3500 reset. When the next power supply starts, the program unit 3100 controls to connect the power supply circuit, thus, the linear actuator 3800 is powered again.

FIG. 5a-FIG. 5c are schematic diagrams of a control circuit of a driving structure according to another embodiment of the present invention.

The power supply 4000, the program unit 4100, the first switch unit 4200 and the linear actuator 4800 are electrically connected to form a power supply circuit. The movement mode of the driving unit 4500 is similar to the foregoing said. As shown in FIG. 5a, the electrical connection point A is located on the power supply circuit between the first switch unit 4200 and the linear actuator 4800. The program unit 4100 is electrically connected to the electrical connection point A to obtain the second signal. Preferably, the second signal is also a voltage signal. Before the driving unit 4500 contacts the electrical contact point 4710, the electrical connection point A is at a low voltage state.

As shown in FIG. 5b, when the driving unit 4500 is in contact with the electrical contact 4710, the triggered first signal (such as a voltage signal) turns off the first switch unit 4200, cutting off the power to the linear actuator 4800, and the electrical contact 4710 returns to the high voltage state. Generally, since the linear actuator 4800 does not immediately withdraw or remove the tension after the power is off (because of the material phase change taking a certain time), the driving unit 4500 and the electrical contact point 4710 will continue to be in contact for a period of time. The voltage of the electrical connection point A will be the same as that of the electrical contact point 4710, that is, maintains a high voltage state. Therefore, before and after the first switch unit 4200 is turned off, the voltage of the electrical connection point A is different, thus a second signal is generated and sent to the program unit 4100. Obviously, in the embodiment of the present invention, the second signal is a voltage changing signal.

As shown in FIG. 5c, after the linear actuator 4800 releases the tension, the driving unit 4500 leaves the electrical contact 4710 under the force of the elastic member, the electrical contact 4710 returns to the initial high voltage state, and the first switch unit 4200 is turned on again. Since the program unit 4100 controls to disconnect the power supply circuit, the linear actuator 4800 still cannot be powered, thus the driving unit 4500 resets. When the next power supply starts, the program unit 4100 controls to connect the power supply circuit, making the linear actuator 4800 powered by the power supply 4000 again.

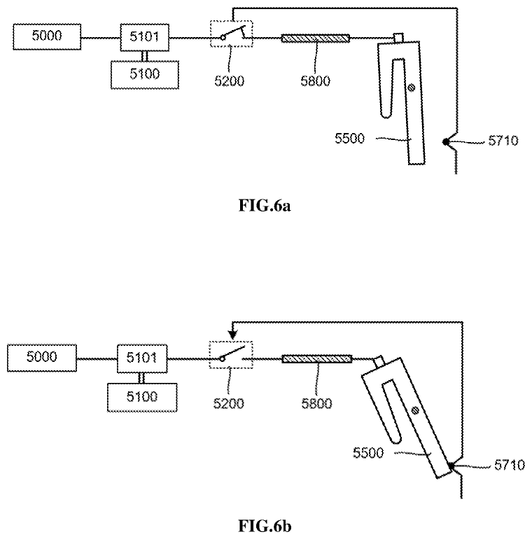

FIG. 6a-FIG. 6b are schematic diagrams of a driving structure control circuit according to another embodiment of the present invention.

As shown in FIG. 6a, in this embodiment, the program unit 5100 includes a timer 5101 for controlling the disconnection and connection of the power supply circuit for supplying power to the linear actuator 5800. After going off a period of time T, the timer 5101 controls to disconnect the power supply circuit. If the time from the linear actuator being powered to triggering the first signal is t, then T≥t. In some embodiments of the present invention, 0 ms≤T−t≤30 ms. In one embodiment of the present invention, T−t=5 ms. In another embodiment of the present invention, T−t=10 ms. In yet another embodiment of the present invention, T−t=25 ms.

As shown in FIG. 6b, when the driving unit 5500 is in contact with the electrical contact point 5710, the triggered first signal turns off the first switch unit 5200, making the linear actuator 5800 stop being powered by the power supply 5000. After the T−t time, the timer 5101 controls the power supply circuit to be disconnected.

As mentioned above, when the driving unit 5500 leaves the electrical contact point 5710, the first switch unit 5200 is turned on again, but for the timer 5101 controls the power supply circuit to be disconnected, the linear actuator 5800 is not powered until the next driving starts.

In the embodiment of the present invention, the response speed of the first switch unit is faster than that of the program unit. Compared with the program unit simultaneously controlling the power on and off of the linear actuator, the first switch unit can cut off the power to the linear actuator more quickly, shortening the time the linear actuator powered, thus reducing the power consumption of the infusion device. In addition, shortening the time that the linear actuator is powered on also reduces the probability of fatigue fracture of the linear actuator, which improves the safety of the drive and the infusion reliability of the infusion device.

Here, the response speed of the first switch unit is faster than that of the program unit means that after receiving the electrical signal, the first switch unit quickly cuts off the power to the linear actuator, so as to prevent the program unit from controlling to cut off the power to the linear actuator. Therefore, the electrical signal or the second signal detected by the program unit is not necessarily used to disconnect the power supply circuit to the linear actuator, but can also be used for other purposes, such as recording the number of times the linear actuator is powered on, which helps to optimize the control process of the program unit.

In summary, the present invention discloses a drive structure of a drug infusion device. The program unit and the first switch unit control the power to the linear actuator, but only the first switch unit cuts off the power to the linear actuator. The response speed of the first switch unit is faster than that of the program unit, reducing the power consumption of the infusion device.

While the invention has been described in detail with reference to the specific embodiments of the present invention, it should be understood that it will be appreciated by those skilled in the art that the above embodiments may be modified without departing from the scope and spirit of the invention. The scope of the invention is defined by the appended claims.

1. A drive structure of a drug infusion device, comprising:
at least one driving unit and at least one driving wheel, wherein the driving unit, moving in a driving direction, drives the driving wheel to rotate;
a linear actuator, electrically connected with the driving unit, pulling the driving unit to move in the driving direction after being powered;
a power supply used to supply power to the linear actuator;
a program unit and a first switch unit, wherein the power supply, the program unit, the first switch unit and the linear actuator are electrically connected to form a power supply circuit, when the linear actuator is powered, the driving unit implements driving, and the driving unit triggers a first signal, indicating an end point of the driving direction, which controls the first switch unit to turn off the power to the linear actuator.

2. A drive structure of a drug infusion device of claim 1, wherein
the driving unit includes at least one driving portion, the driving wheel is provided with wheel teeth which are pushed by the driving portion to drive the driving wheel.

3. A drive structure of a drug infusion device of claim 2, wherein
a movement mode of the driving unit includes a linear reciprocating movement or a rotary reciprocating movement.

4. A drive structure of a drug infusion device of claim 3,
further comprising an elastic member, which applies a resetting and resilience force to the driving unit, wherein the elastic member cooperates with the linear actuator to make the driving unit reciprocate.

5. A drive structure of a drug infusion device of claim 4,
further comprising an electrical contact point, which is used to determine the end point of the movement of the driving unit in the driving direction, wherein the driving unit contacts the electrical contact point to trigger the first signal which is an electrical signal.

6. A drive structure of a drug infusion device of claim 5, wherein
the electrical contact point is electrically connected with the first switch unit to form a connection circuit, and the electrical signal controls the first switch unit to turn off.

7. A drive structure of a drug infusion device of claim 5,
further comprising a controller, which is electrically connected to the first switch unit and the electrical contact point, wherein the electrical signal controls the first switch unit to turn off through the controller.

8. A drive structure of a drug infusion device of claim 6, wherein
before and after the driving unit contacts the electrical contact point, voltages of the electrical contact point are different, and the electrical signal is a voltage changing signal.

9. A drive structure of a drug infusion device of claim 5, wherein
the program unit is electrically connected with the electrical contact point to receive the electrical signal.

10. A drive structure of a drug infusion device of claim 9, wherein
the program unit includes a timer which controls to disconnect the power supply circuit, and when the linear actuator starts to be powered, the timer starts timing, after the timer goes off a period of time T, if the time from the linear actuator being powered to triggering the first signal is t, then T≥t.

11. A drive structure of a drug infusion device of claim 10, wherein
0 ms≤T−t≤30 ms.

12. A drive structure of a drug infusion device of claim 10,
further comprising a second switch unit, which is arranged on the power supply circuit, and controlled to disconnect the power supply circuit by the program unit.

13. A drive structure of a drug infusion device of claim 4,
further comprising an electrical connection point located on the power supply circuit between the linear actuator and the first switch unit, wherein the program unit is electrically connected to the electrical connection point to obtain a second signal, a voltage changing signal, of the electrical connection point.

14. A drive structure of a drug infusion device of claim 3,
further comprising a pressure sensor, wherein the pressure sensor is used to determine the end point of the movement of the driving unit in the driving direction, and the first signal is a pressure changing signal.

15. A drive structure of a drug infusion device of claim 12, wherein
the first switch unit or the second switch unit includes a MOS field effect transistor, an analog switch or a relay.

16. A drive structure of a drug infusion device of claim 1, wherein
the linear actuator is a shape memory alloy.

17. A drive structure of a drug infusion device of claim 7, wherein before and after the driving unit contacts the electrical contact point, voltages of the electrical contact point are different, and the electrical signal is a voltage changing signal.
